(19) United States
(12) Patent Application Publication
(10) Pub. No.: US 2013/0317393 A1
(43) Pub. Date: Nov. 28, 2013

(54) PRESSURE SENSOR ASSEMBLY AND ASSOCIATED METHOD FOR PREVENTING THE DEVELOPMENT OF PRESSURE INJURIES

(71) Applicants: Dan Weiss, Ramat Hasharon (IL); Ruth Poliakine-Baruchi, Tel Aviv (IL); Amir Ben Shalom, Modiin (IL); Lior Greenstein, Tel Aviv (IL); Yonatan Assouline, Kfar Bilu (IL); Itai Raab, Hod Hasharon (IL); Boaz Ben David, Hod-Hasharon (IL); Tal Remez, Jerusalem (IL); Gusti Averbuch, Modiin (IL); Efrat Zaritzky, Tel Aviv (IL)

(72) Inventors: Dan Weiss, Ramat Hasharon (IL); Ruth Poliakine-Baruchi, Tel Aviv (IL); Amir Ben Shalom, Modiin (IL); Lior Greenstein, Tel Aviv (IL); Yonatan Assouline, Kfar Bilu (IL); Itai Raab, Hod Hasharon (IL); Boaz Ben David, Hod-Hasharon (IL); Tal Remez, Jerusalem (IL); Gusti Averbuch, Modiin (IL); Efrat Zaritzky, Tel Aviv (IL)

(73) Assignee: ENHANCED SURFACE DYNAMICS, INC., Wellesley, MA (US)

(21) Appl. No.: 13/881,169

(22) PCT Filed: Oct. 26, 2011
(86) PCT No.: PCT/IB11/54773

§ 371 Date: Aug. 6, 2013

Related U.S. Application Data
(60) Provisional application No. 61/407,563, filed on Oct. 28, 2010.
(60) Provisional application No. 61/410,055, filed on Nov. 4, 2010.

(51) Int. Cl. A61B 5/00

(52) U.S. Cl. CPC A61B 5/445; A61B 5/7275; A61B 5/4836; A61B 5/0004; A61B 5/742; A61B 5/746
USPC 600/587

(57) ABSTRACT

A pressure wound prevention system comprising at least one management device which comprises at least one user input apparatus, at least one output mechanism, at least one data input for connecting with a pressure sensor assembly and a processor configured to measure elapsed time, to receive pressure data from the data input and to present an output via the output mechanism. The system may be modular. A method for preventing the development of pressure wounds of a subject is also presented.

### FIELD OF THE INVENTION

The embodiments disclosed herein relate to a pressure sensing apparatus. In particular, devices and methods are disclosed for managing and controlling pressure sensing in pressure wound prevention systems.

### BACKGROUND

Pressure wounds e.g. decubitus ulcers, which are commonly known as pressure ulcers or bedsores, are lesions developed when a localized area of soft tissue is compressed between a bony prominence and an external surface for a prolonged period of time. Pressure ulcers may appear in various parts of the body, and their development is affected by a combination of factors such as unrelieved pressure, friction, shearing forces, humidity and temperature.

Currently, about 10%-15% of hospitalized patients are estimated to have bedsores at any one time (Source: Medicare website 2009). However, it is not only hospitalized patients who suffer from pressure wounds: for example, people confined to wheelchairs are prone to suffer from pressure wounds, especially in their pelvis, lower back and ankles. Although easily prevented and completely treatable if found early, bedsores are painful, and treatment is both difficult and expensive. In many cases bedsores can prove fatal—even under the auspices of medical care.

The most effective way of dealing with pressure wounds is to prevent them. Existing preventive solutions are either passive (e.g. various types of cushioning) or active, including a range of dynamic mattresses that alternate the inflation/deflation of air cells. Typically, such mattresses re-distribute pressure even from unnecessary locations thereby needlessly creating higher pressure in sensitive areas. Moreover, such mattresses are typically designed for patients lying down in hospital beds, and hardly answer the needs of individuals who spend considerable amounts of time sitting up, confined to a wheelchair or the like.

The most common preventive approach is keeping a strict routine of relieving pressure from sensitive body areas of a patient every 2-3 hours. This can be done with patients under strict medical care. Apart from being a difficult, labor intensive and costly task, it does not meet the needs of independent individuals who do not require ongoing supervision of a caretaker, such as paraplegics who use a wheelchair for mobility.

### SUMMARY

There is an established need for a reliable, cost effective system and method for preventing pressure wounds from forming. Various systems are disclosed herein addressing this need.

According to one aspect of the disclosure, a pressure wound prevention system comprising: at least one management device, comprising at least one user input apparatus, at least one output mechanism, and at least one data input for connecting with a pressure sensor assembly; and a processor configured to measure elapsed time, to receive pressure data from the data input and to present an output via the output mechanism.

The processor may be configured to use the pressure data to calculate a risk index. Optionally, the processor may be configured to compare calculated risk index values with at least one threshold value. Variously, the at least one threshold value is enterable via the user input. Alternatively or additionally, the at least one threshold value is calculated by the processor.

As required, the risk index may comprise an elapsed time value. Furthermore the risk index may comprise an accumulated pressure value. For example, the accumulated pressure value may comprise a product of a pressure measurement and a duration for which the pressure measurement is recorded. Where required, a clock-reset button may be operable to reset elapsed time value to zero.

In various pressure wound prevention systems the processor may be configured to receive settings via the user input. Such settings may be selected from at least one of a group consisting of: threshold accumulated pressure, maximum elapsed time, user details, subject details, medical condition of subject, and combinations thereof.

Optionally, the processor may be configured to send at least one alert signal to the output mechanism when the calculated risk index is greater than the at least one threshold value. Accordingly, the output mechanism is configured to display a first warning when the calculated risk index is greater than the first threshold value. Additionally, or alternatively, the output mechanism may be configured to display a second warning when the calculated risk index is greater than a second threshold value.

In a particular version of the pressure wound prevention system the pressure sensor assembly comprises a plurality of pressure sensor elements. Accordingly, the processor may be configured to receive pressure data associated with each pressure sensor element. The processor may then be configured to use the pressure data to calculate a plurality of risk indices, where each risk index may be associated with a set of pressure sensor elements. In such pressure wound prevention systems the processor may be configured to compare calculated risk index values with threshold values for each set of pressure sensor elements.

Optionally, at least one set of pressure sensors is selected to correspond to a section of a recorded subject. For example, where the recorded subject comprises a living body, the section may comprise a region of the living body at risk of developing a pressure wound.

Where required, the output mechanism may be operable to display a pressure map relating to pressure readings received from the plurality of pressure sensor elements. Additionally or alternatively, the output mechanism may be operable to display a map relating to accumulated pressure readings received from the plurality of pressure sensor elements. Optionally, the output mechanism may be operable to display a map representing risk indices calculated for a plurality of regions of a subject.

Variously, the output mechanism may comprise at least one of a group consisting of: alarms, visual displays, audio displays, buzzers, remote units and combinations thereof.

Optionally, the pressure wound prevention system may further comprise at least one transmitter configured to communicate output signals to a receiver associated with a remote unit. Additionally, the pressure wound prevention system may further comprise at least one receiver configured to receive input signals from a transmitter associated with a remote unit.

Accordingly, the pressure wound prevention system may further comprise a remote unit configured to present the output to a remote user. Optionally, at least one the user input apparatus may comprise the remote unit. In various systems, a remote unit may be configured to present output from a plurality of the management devices.

Variously, the user input apparatus may comprise at least one of a group consisting of: touchscreens, keypads, mice, touchpads, roller-balls and combinations thereof.

In another aspect of the disclosure, a modular pressure wound prevention system is presented comprising at least one sensor module configured to sense pressure measurements wherein the sensor module is further configured to communicate with at least one management module. Optionally, the sensor module may comprise a plurality of pressure sensor elements.

Furthermore, the sensor module comprises at least one layer of insulating material sandwiched between a first conductive layer and a second conductive layer. Optionally, the sensor module may comprise a protective cover and/or an overlay sheet.

In one example, the sensor module comprises: a layer of an insulating material sandwiched between a first set of parallel conductive strips and a second set of parallel conductive strips arranged orthogonally to the first set of parallel conductive strips; a protective cover; and a docking station configured to connect with the management module.

The sensor module may comprise a docking station configured to connect to at least one management module. The docking station may comprise at least one set of electrical contacts configured to connect with a second set of electrical contacts of at least one management module. Additionally or alternatively, the docking station comprises a mechanical connector configured to mechanically couple at least one management module to the sensor module.

The sensor module may have a shorter lifespan than the management module. Accordingly, the sensor module may further comprise a data storage unit configured to store historical data relating to the sensor module.

Optionally, the sensor module comprises a wireless communicator configured to communicate pressure data to the management module. Where required, the sensor module comprises a power supply.

In another aspect of the disclosure, a modular pressure wound prevention system is presented comprising at least one at least one management module configured to receive pressure data from a sensor module and to present an output to a user.

Optionally, the management module may comprise a mechanical connector configured to mechanically couple the management module to the sensor module. Additionally, or alternatively, the management module may comprise at least one set of electrical contacts configured to connect with a second set of electrical contacts of the sensor module.

In some examples, the management module may comprise a hardware controller configured to provide analog control to the sensor module. Optionally, the management module may comprise a system control unit configured to receive pressure data from the sensor module and to present an output to a user.

In still another aspect of the disclosure, a method is taught for preventing the development of pressure wounds of a subject. The method may comprise: providing a pressure sensor assembly; providing a processor configured to measure elapsed time and to receive pressure data from the pressure sensor assembly; calculating a risk index; comparing the risk index with at least one threshold value; and the processor sending an alert signal to an output mechanism if the risk index is greater than the threshold value. Optionally, the method further includes displaying a map of the risk index.

The method may further comprise: providing at least one sensor module; providing at least one management module; connecting the management module to the sensor module; the sensor module measuring pressure exerted by the subject; the management module receiving pressure data from the sensor module; and the management module presenting an output to a user indicating risk of subject developing pressure wounds.

In various examples, the step of calculating a risk index may comprise: defining a risk index function r(τ,x,y) relating pressure exerted upon the subject to the risk of the subject developing a pressure wound; measuring pressure exerted by the subject upon a plurality of pixels upon a pressure sensor assembly; measuring the time elapsed during which each pixel records the pressure; calculating the value of the risk index function for each pixel.

Accordingly the method may further comprise providing medical data pertaining to the subject. For example the medical data may be selected from a group consisting of: the region of the body at which the pressure is measured, age of subject, weight of subject, gender of subject, medical condition of subject and combinations thereof.

Variously, the risk index function may be dependent upon at least one parameter selected from a group consisting of: measured pressure, time elapsed, coordinates of pixel recording pressure, the region of the body at which the pressure is measured, age of subject, weight of subject, gender of subject, medical condition of subject and combinations thereof.

It is noted that the risk index function may be represented by the formula:

r(τ,x,y)=Σₜ₌₁^{τ}K(t−τ)*p(P(τ,x,y))*sᵤ(x,y,w,a)

Optionally, the method may further comprise mapping each the plurality of pixels to a point on the body of the subject. The mapping may comprise: providing a body based coordinate system; identifying the posture of the subject; and associating each the pixel with a point in the body based coordinate system.

Optionally, the step of identifying the posture of the subject may comprise: obtaining a set of reference pressure images corresponding to a set of reference postures; recording measured values of pressure exerted by the subject upon each the pixel; obtaining a pressure image of the subject; comparing the pressure image of the subject with the reference pressure images; and selecting a reference posture corresponding to the obtained pressure image.

### BRIEF DESCRIPTION

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

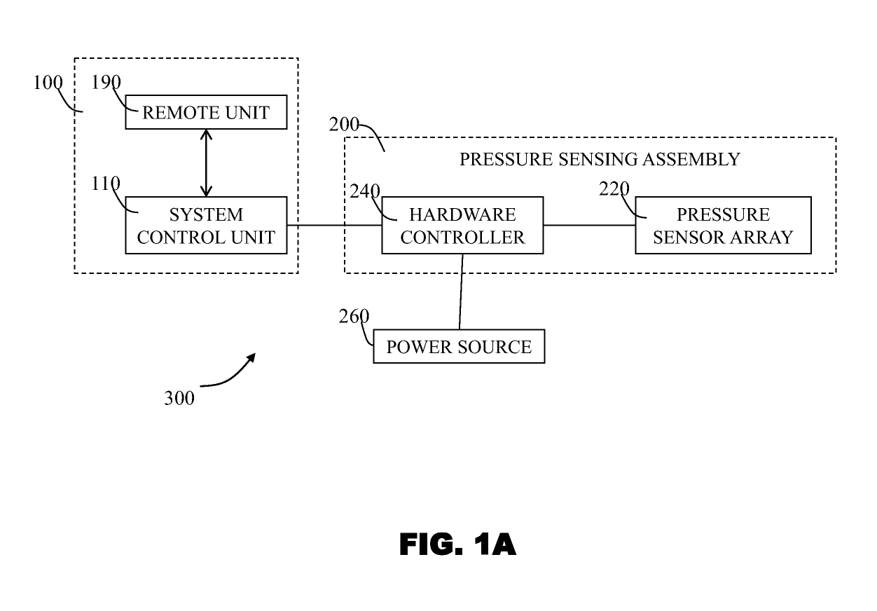

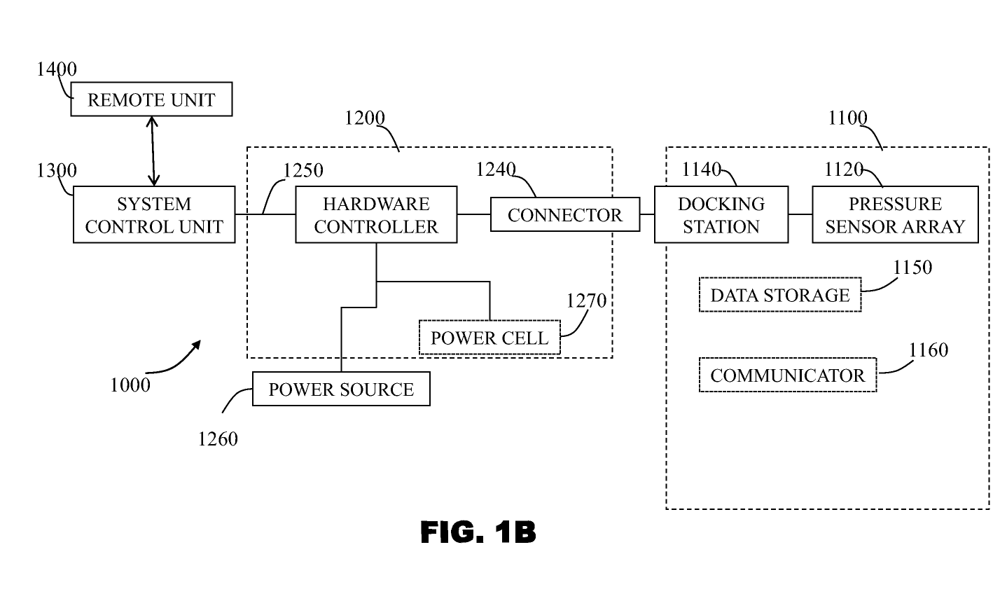

FIG. 1A is a block diagram showing the main elements of an injury prevention system incorporating an example of a management device and a pressure sensing assembly;

FIG. 1B is a block diagram showing the main elements of an example of the injury prevention system comprising a modular pressure wound prevention system;

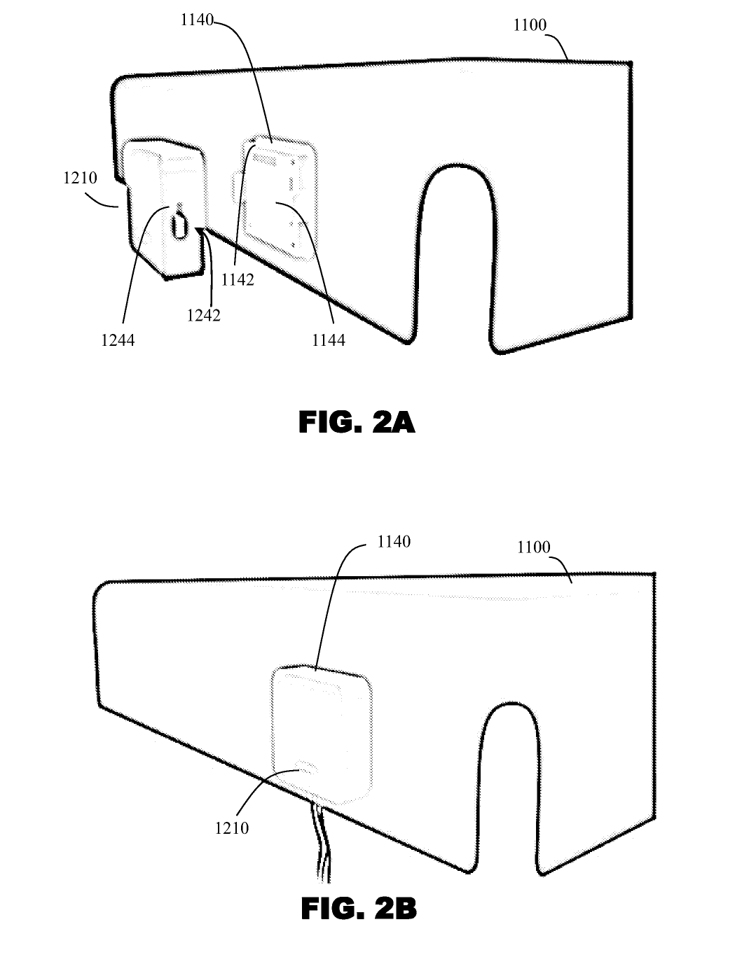

FIG. 2A is a schematic representation of an example of a hardware control module and an example of a sensor module;

FIG. 2B is a schematic representation of the hardware control module of FIG. 2A docked to the sensor module of FIG. 2B;

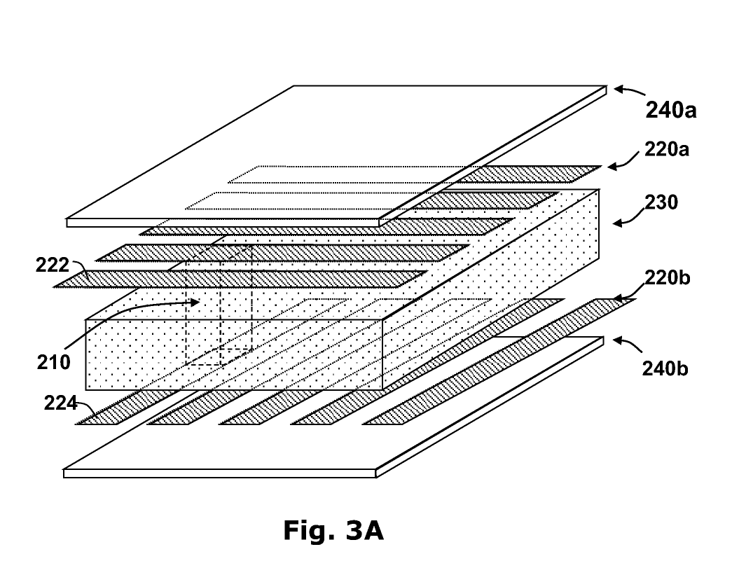

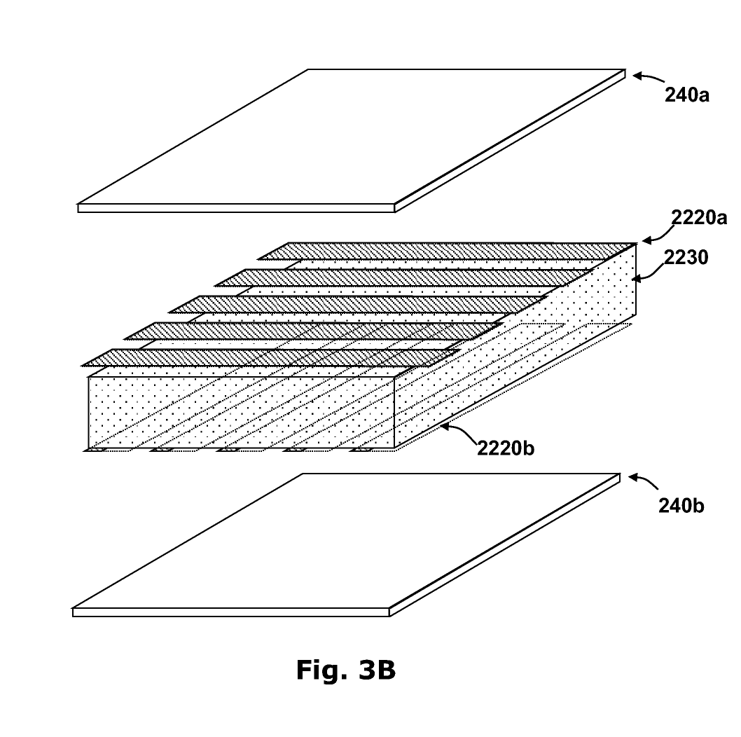

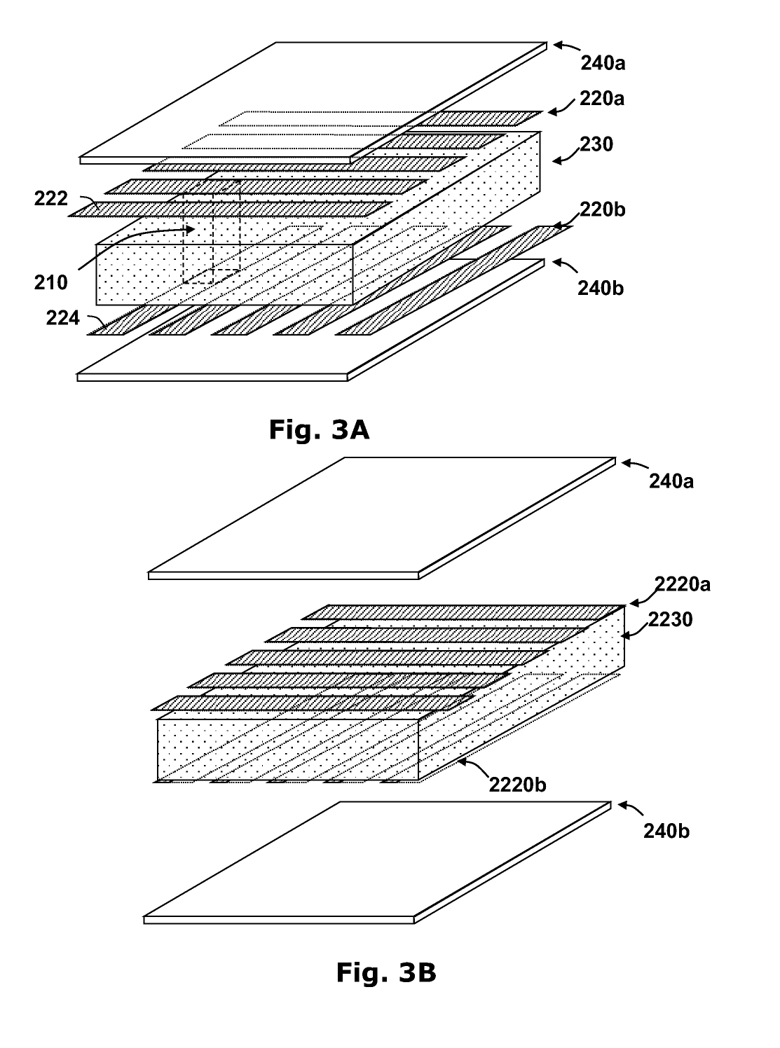

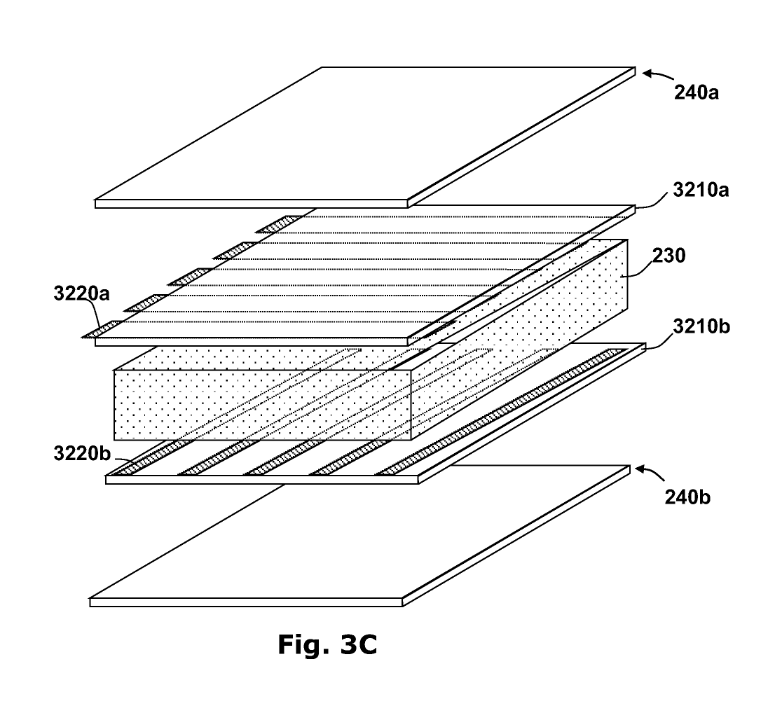

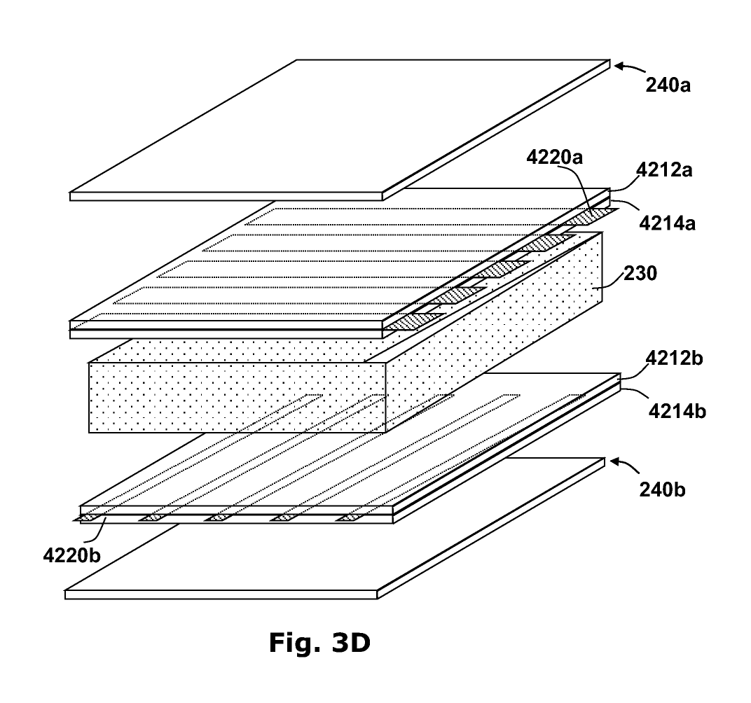

FIG. 3A-D show isometric projections of various possible examples of a pressure detection mat for use in a sensor module;

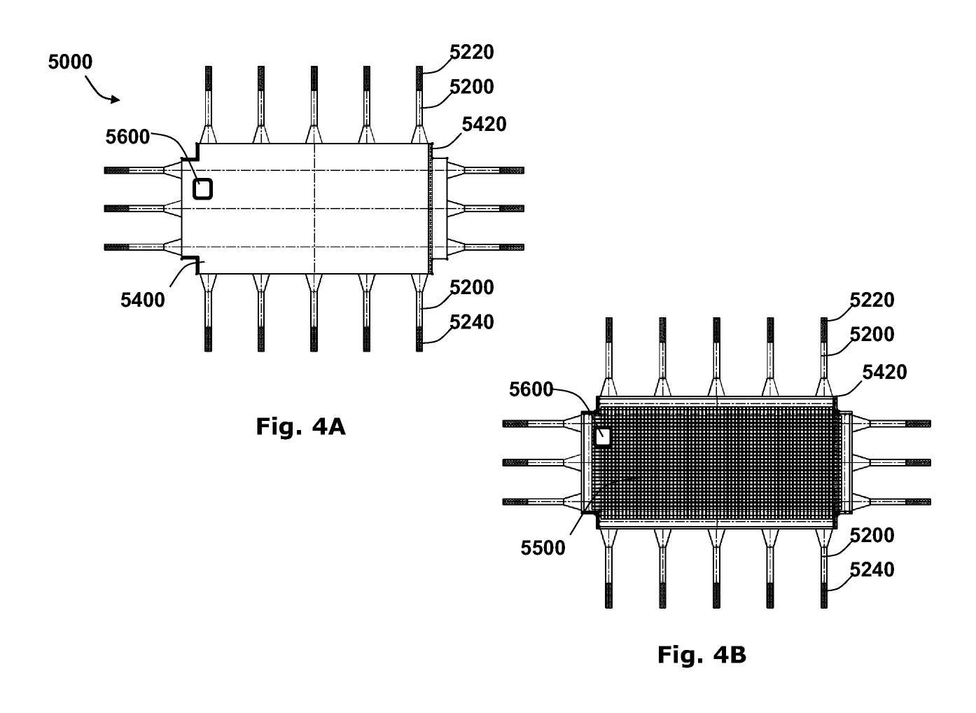

FIGS. 4A and 4B are a top view and section through respectively are shown of a further example of a sensor module incorporated into a mattress overlay;

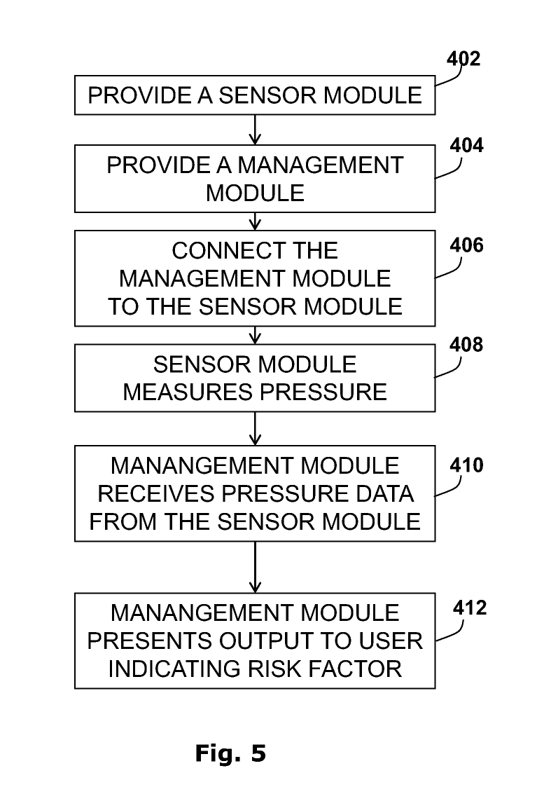

FIG. 5 is a flowchart of a method for using a modular pressure wound prevention system;

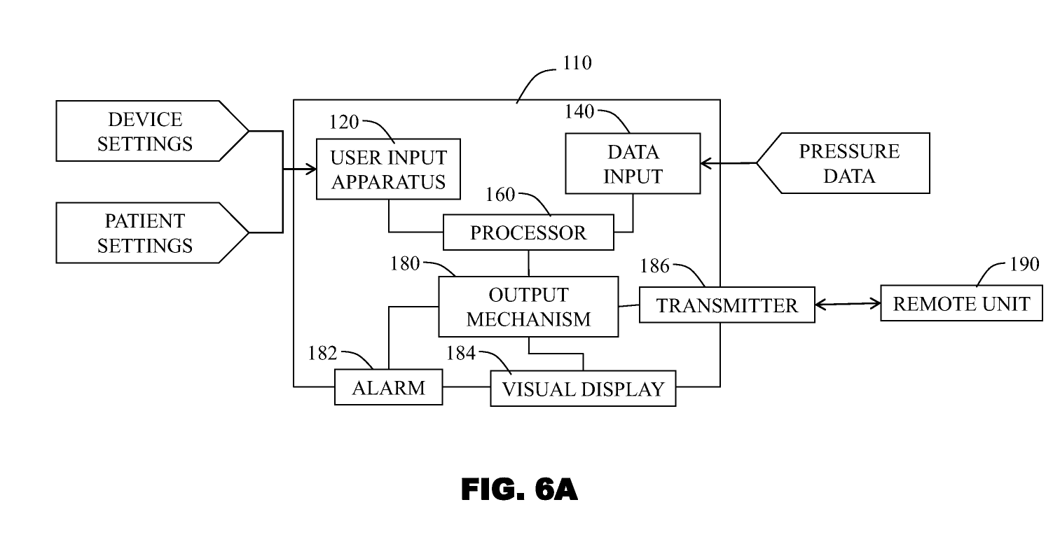

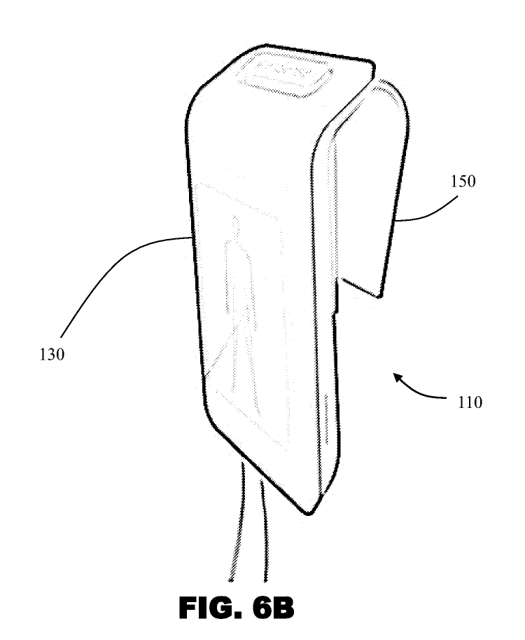

FIG. 6A is a block diagram representing the main elements of a management device for an injury prevention system;

FIG. 6B is a schematic diagram of an example of a system control unit for the injury prevention system;

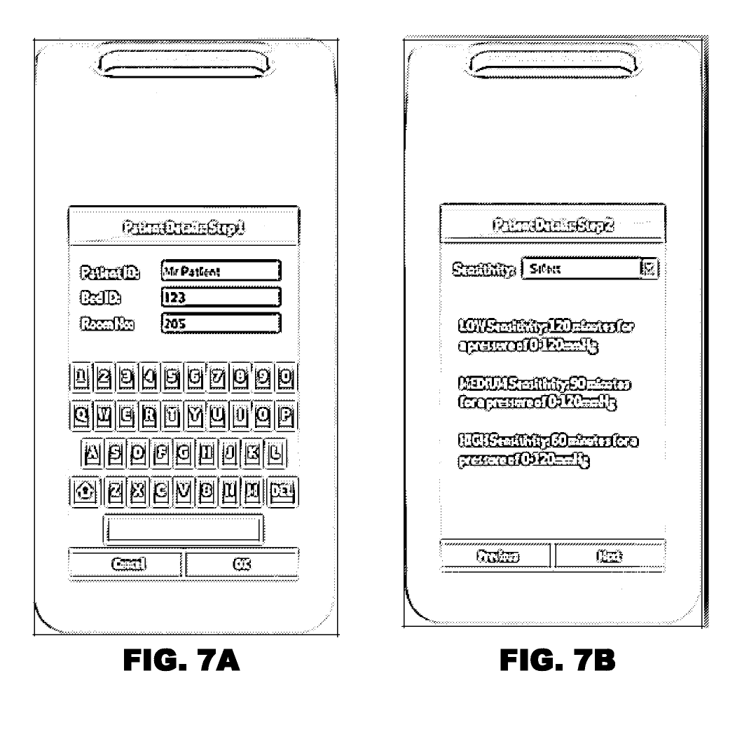

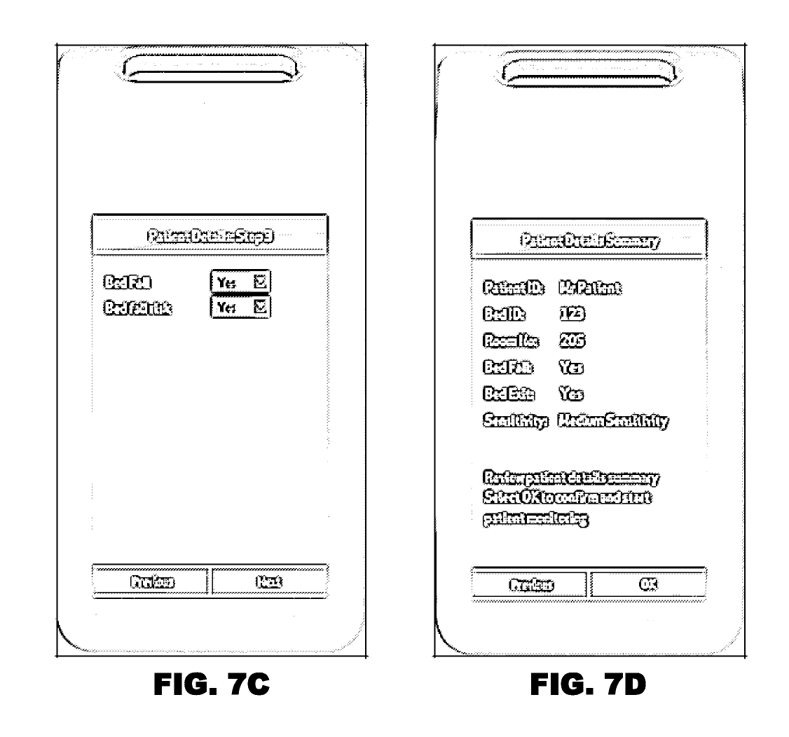

FIGS. 7A-D are screenshots of possible settings screens which may be used during configuration of the system;

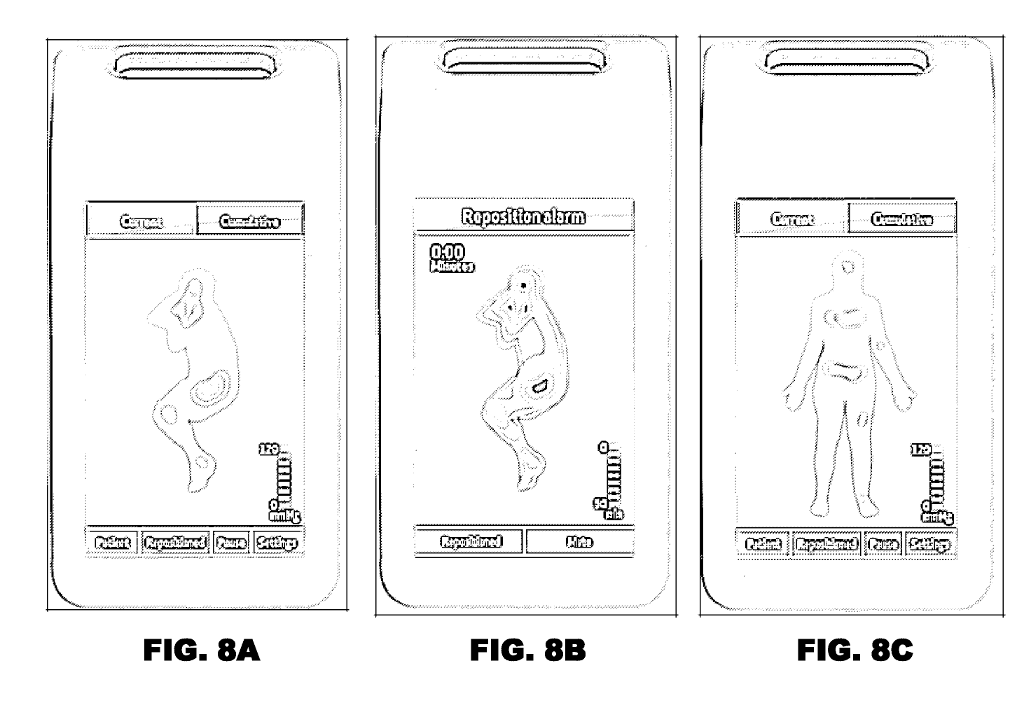

FIGS. 8A-C are screenshots of possible output screens displaying maps representing pressure data collected by the pressure sensor assembly;

FIG. 9 is a screenshot of a possible output screen of a remote device for monitoring a plurality of subjects;

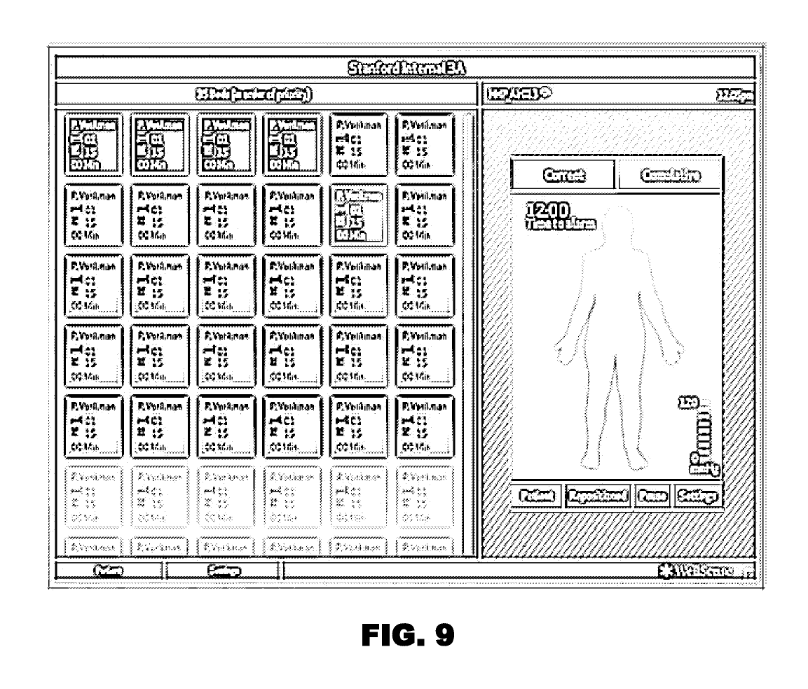

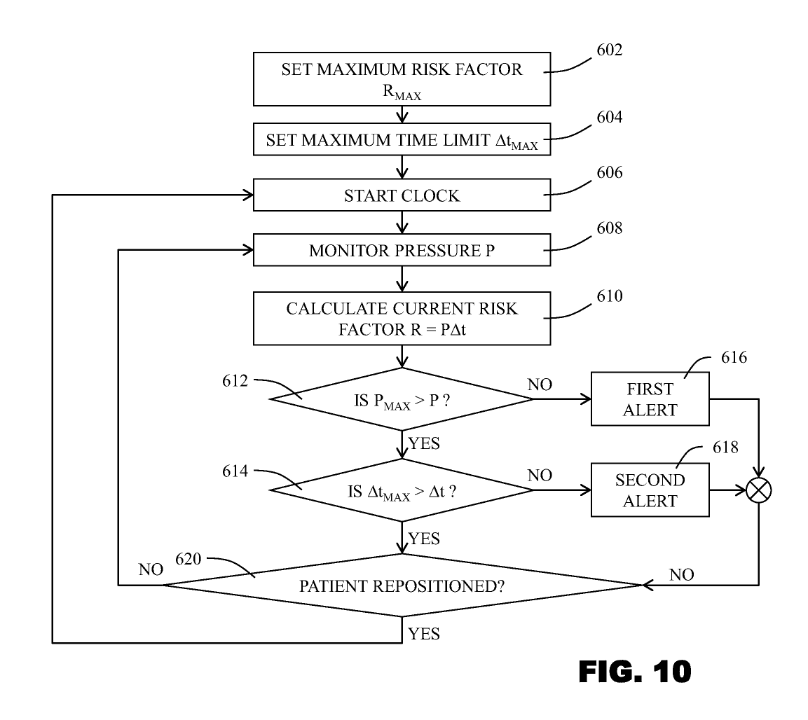

FIG. 10 is a flowchart showing illustrating a possible method for controlling the output of the management device; and

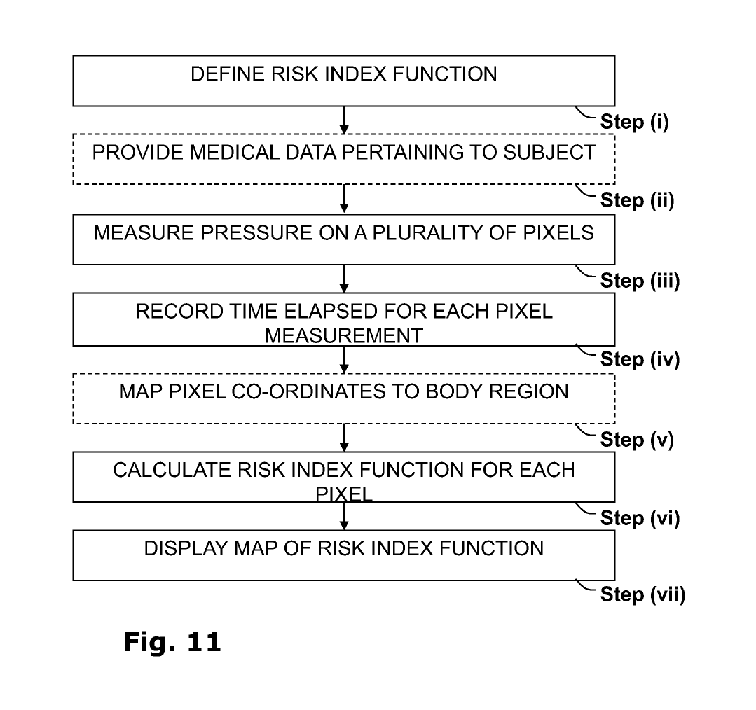

FIG. 11 is a flowchart showing another method for presenting pressure data related to the risk of a subject from developing pressure injuries.

### DETAILED DESCRIPTION

Reference is now made to the block diagram of FIG. 1A showing the main elements of an injury prevention system 300 incorporating an example of a management device. The injury prevention system 300, such as a pressure wound prevention system for example, may include a pressure sensor assembly 200 and a management system 100 and may be used to prevent the development of pressure related injuries such as decubitus ulcers or the like.

The pressure sensor assembly 200 is provided to measure the pressure exerted upon a subject over time. The pressure sensor assembly 200 includes a pressure sensor array 220 and a hardware controller 240. The hardware controller 240 may be configured to provide power and analog control to the pressure sensor array 220. The hardware controller 240 may be further configured to transfer output signals from the pressure sensor assembly 200 to the management system 100.

According to a particular example, the pressure sensor array 220 may be a pressure sensing mat configured to be placed between a mattress and the body of a bed-bound patient, for example. Other examples of the pressure sensor array 220 may include pressure sensitive pads, cushions, clothing or the like.

The management system 100 includes a system control unit 110, provided to control the settings and operation of the pressure sensor assembly 200 as well as to provide output from the system to a user. Optionally the system control unit 110 is in communication with a remote unit 190 enabling a user to configure settings and monitor the output of the system remotely. It will be appreciated that a remote unit 190 may be further configured to communicate the output from a plurality of system control units 110 such the remote user may be able to monitor a plurality of subjects. This may be of particular use, for example, in a hospital setting, where a bedside system control unit 110 may be configured to communicate with a remote unit 190 at a nurses' station for example. The remote unit 190 may be further configured to record data in a storage device for subsequent retrieval.

Reference is now made to the block diagram of FIG. 1B showing an example of a modular system 1000 which may be used to prevent pressure wound formation. The system 1000 includes a sensor module 1100 and two management modules: a hardware controller module 1200 and a system control module 1300. Optionally the system 1000 may further include a remote unit 1400 in communication with the system control unit 1300 and via which the system 1000 may be controlled and its output monitored.

The pressure wound prevention system 1000 may be used to monitor pressure distribution between a subject and a surface and to alert a carer to potential risk of the subject developing pressure wounds. Using such a pressure wound prevention system may therefore enable the carer to take preventative action such as turning or otherwise repositioning the subject before pressure wounds develop.

It is a particular feature of the modular system 1000 that the various modules may be exchanged or replaced independently from one another. This may be an important aspect of systems in which different modules have different lifespans and a shorter lifespan module may be replaced without exchanging modules with longer lifespans.

The sensor module 1100 includes a pressure sensor array 1120 and a docking station 1140. The array of pressure sensors 1120 is configured to measure pressure between the subject and a surface. The docking station 1140, as described in greater detail below, may provide a communication channel between the sensor array and the management modules 1200, 1300.

The sensor module 1100 may further include a number of additional elements such as a data storage unit 1150, communicator 1160 or integrated power cell 1170, for example. The data storage unit may be configured to store historical data relating to the sensor module 1100 for use in calculation of risk factors. Such data may, for example, include records of initial values of certain reference parameters as well as their variation with time so as to provide corrections to pressure measurements.

It will be appreciated that the inclusion of a data storage unit 1150 such as a memory chip or the like may enable the sensor module 1100 to be readily connected to different management modules with a continuity of record being maintained. Thus, for example, in a hospital environment a bed-bound patient is often moved from one ward to another while remaining in the same bed. The sensor module 1100, such as the overlay sheet described hereinbelow, may be disconnected from a first management module in the first ward and reconnected to add a new management module in a new ward with no loss of continuity to the data record.

A communicator 1160 unit may be provided to communicate data to the management modules. Where appropriate, wireless communicators may provide a communication channel via transceivers such as radio transceivers, inductive transfer systems or the like, possibly using known protocols such as WiFi, Bluetooth, NFC or the like.

The hardware controller module 1200 is configured to receive analog sensor signals from the sensor module 1100 via a connector 1240 coupled to the docking station 1140. Analog sensor signals received from the sensor module 1100 may be transferred to the system control unit 1300. Additionally, the hardware controller module 1200 may provide power to the sensor module 1100, possibly from an external power source 1260 or an internal power cell 1270. Alternatively, the sensor module 1100 may comprise its own on board power cell 1170 such as a battery pack or the like.

The system control unit 1300 is provided to control the settings and operation of the system 1000 as well as to provide output from the system to a user. The system control unit 1300 may be connected to the hardware controller 1200 via a communication line 1250. It is noted that the communication line may be a wired communication cable, a wireless link or such like. It is particularly noted that in hospital settings, where equipment such as beds and the like are often moved around, a robust communication cable is desirable, accordingly an extendable, coiled, helical or other cable may be used such that it tends to extend rather than to break when snagged.

Optionally the system control unit 1300 is in communication with a remote unit 1400 enabling a user to configure settings and monitor the output of the system 1000 remotely.

The docking station 1140 is configured to couple with a connector 1240 of the hardware controller 1200 and may provide a communication channel between the sensor array and the management modules 1200, 1300. In addition the docking station 1140 may provide a power line to connect the sensor array 1120 to a power source 1260.

Reference is now made to FIGS. 2A and 2B schematically representing an example of a hardware control unit 1200 configured to connect with the docking station 1140 for use with a modular pressure wound prevention system 1000. With particular reference to FIG. 2A, the docking station 1140 is mounted upon an overlay sheet 1110 housing a pressure sensor array 1120. The docking station 1140 includes a first set of electrical connectors 1142 and a first set of mechanical connectors 1144. The hardware control unit 1200 of the example includes a connection box 1210 having a second set of electrical connectors 1242 and a second set of mechanical connectors 1244.

As represented in FIG. 2B, the connection box 1210 may be pressed to the docking station 1140 such that the first set of mechanical connectors 1144 engages the second set of mechanical connectors 1244. Thus the hardware control unit module 1200 may be secured firmly to the sensor module 1100. The electrical connectors 1122, 1142 are positioned such that when the connection box 1210 is engaged, the first set of electrical connectors 1142 connect with the second set of electrical connectors 1242 providing a data channel between the modules.

The pressure sensor array 1120 may be a pressure sensing mat placed between a mattress and the body of a bed-bound patient, for example. Other examples of the pressure sensor array 1120 may include pressure sensitive pads, cushions, clothing or the like.

Reference is now made to FIG. 3A showing an isometric projection of an example of a pressure-detection mat 200, such as described in the applicants co-pending PCT application no. PCT/IL2010/000294, which comprises a plurality of sensors 210 arranged in a form of a matrix. The mat of the example has two layers 220a, 220b of conductive material separated by an insulating layer 230 of isolating material. Each of the conductive layers typically consists of parallel conductive strips 222, 224 and the two conductive layers are arranged orthogonally such that in one conductive layer the strips are horizontal 222 and in the other conductive layer they are vertical 224. Each strip is wired to a control unit and is preferably operable by a safe low voltage source.

A capacitance sensor is based on the capacitance between the sections of the conducting strips overlapping at each “intersection” of a vertical conductive strip with a horizontal conductive strip. These capacitance sensors are configured such that pressing anywhere over their surface changes the spacing between the two conductive layers, and consequently the capacitance of the intersection. A driving unit may selectively provide an electric potential to the vertical strip and the electrical potential may be monitored on the horizontal strip such that the capacitance sensor of the overlapping section may be determined.

It is noted that by providing an oscillating electric potential across each sensor and monitoring the alternating current produced thereby, the impedance of the intersection may be calculated and the capacitance of the intersection determined. Thus, where the mechanical properties of the sensor are known, the pressure applied upon the sensor may be deduced.

A capacitance sensor may retain its functionality even if it is fully pressed continuously for extended periods, keeping its characteristics even after sustained use over periods even longer than a year with sensor characteristics being consistent between two separate events. The mat may further include additional sensors configured to monitor additional factors, particularly additional factors influencing the development of bedsores, such as temperature, humidity, or the like. Such additional sensors may be configured to monitor the factors continuously or intermittently as appropriate to detect high risk combinations of factors. Such measurements may be recorded and stored in a database for further analysis.

It will be appreciated, however, that due to wear and tear, the performance of the sensing mat may deteriorate over time and it may be necessary for the sensor module to be replaced periodically. It is a particular feature of the modular pressure wound prevention system 1000 (FIG. 1) disclosed herein that the sensor unit 1100 may be replaced without the necessity of exchanging the management modules.

The materials used in the pressure-detection mat may be selected such that the conductive layers and insulating layers are flexible. The insulation material may be a compressible, typically sponge-like, airy or poriferous material (e.g. foam), allowing for a significant change in density when pressure is applied to it. Materials comprising the sensing mat are typically durable enough to be resistant to normal friction of daily use. Furthermore, the sensing mat may be configured so as not to create false pressure readings for example when the mat is folded over onto itself, for example.

The pressure-detection mat 200, or sensing-mat, may be placed underneath or otherwise integrated with other material layers 240a, 240b such as used in standard bed sheets. It will be appreciated that the inclusion of such additional materials may confer further properties as may be required for a particular application. For example, where appropriate, the conductive material of the sensors may be covered by an isolating, washable, water resistant, breathing cover mat, allowing minimum discomfort to the subject resting on the mat.

With reference now to FIGS. 3B-D showing exploded views of sections of various embodiments of the pressure-detection mat, the conductive layers 220 (FIG. 3A) may be supported by various substrates. For example FIG. 3B shows two conductive layers 2220a, 2220b adhered directly to the insulating layer 230. Alternatively, as shown in FIG. 3C, conductive layers 3220a, 3220b may be supported by separate substrates 3210a, 3210b, such as of TPU for example, the insulating layer 230 being sandwiched therebetween. In still another embodiment, as shown in FIG. 3D, the conductive layers 4220a, 4220b may themselves each be sandwiched between two substrates 4212a, 4214a, 4212b, 4214b respectively.

With reference to FIGS. 4A and 4B, a top view and section through are shown respectively of a further example of a sensor module incorporated into a mattress overlay 5000. The overlay 5000 incorporates a sensor matrix 5500 and docking station 5600, such as described hereinabove. The sensor matrix 5500 is housed within a cover sheet 5400 and which may be sealed by a zipper 5420 or alternatively sewn into the cover as required. The sensor module may be connected to a hardware controller (not shown) via the docking station 5600.

The pressure detection mat 5000 may be attached to a surface in such a way that prevents movement of the mat relative to the surface. A feature of the embodiment of the mat 5000 is that the cover mat 5500 may include a coupling mechanism for securing the mat to a seat or a back of a mattress, a bed, a chair, a bench, a sofa, a wheelchair or the like. The coupling mechanism may include for example at least one strap 5200 having an attachment means 5240 configured to secure the straps 5200 to the seat or to each other such that the pressure detection mat is held securely. This may be useful to prevent folding, wrinkling or other movement of the detection mat which may contribute to the creation of shear forces which are known to encourage the formation of external pressure sores. Suitable attachment means include for example, hook-and-eye materials such as Velcro®, buckles, adhesives, buttons, laces or such like as suit requirements.

Referring to the flowchart of FIG. 5 a method is disclosed for the prevention the development of pressure wounds. The method includes the steps: providing a sensor module 402; providing a management module 404; connecting the management module to the sensor module 406; the sensor module measuring pressure exerted by the subject 408, the management module receiving pressure data from the sensor module 410; and the management module presenting an output to a user indicating risk of subject developing pressure wounds 412.

Referring now to the block diagram of FIG. 6A, the main elements are shown of the system control unit 110 used to control the injury prevention system 300 (FIG. 1). The system control unit 110 may include a user input apparatus 120, a data input 140, a processor 160 and an output mechanism 180.

Referring to FIG. 6B, a schematic diagram is presented showing an example of a system control unit 110 for use with the management device of a pressure monitoring system 300. In this example the user interface includes a touch screen 130 which serves both as a user input device and as a visual output device. The interface may display, inter alia, a pressure map of a subject. It is further noted that the system control unit 110 may be provided with an attachment means 150 for mechanically coupling the unit to a bed, an infusion support pole or the like such that it may be conveniently accessed and its user interface displayed.

Referring back to FIG. 6A, the user input apparatus 120 provides a channel via which a user may enter configuration settings to the system. Thus, for example, a carer may be able to enter data relating to the subject being monitored such as name, bed, ward, carer ID and other identification information, as well as details relating to the type of monitoring required to be undertaken. In addition, the input apparatus may provide a means via which medically significant details about the subject, such as age, gender, condition and the like, may be provided. Additional medical details may be used to assign a subject to a suitable risk group which may be used to determine risk indices and threshold values.

The data input 140 is in communication with the pressure sensor assembly and is configured to receive monitoring data therefrom. In particular, the data input 140 is a channel via which pressure related data may be relayed to the processor 160. Apart from pressure data, the data input 140 may be further configured to receive other data such as temperature, humidity or the like, which may be relevant to the assessment of a subjects risk of developing pressure sores or to the calculation of pressure.

FIGS. 7A-D show screenshots of possible settings-input screens which may be used during configuration of the system. During configuration, patient details and risk levels may be set. It will be noted that a touch screen interface may provide a convenient input apparatus as it allows a large output display to use a relatively small module without being compromised by an input device. Alternatively or additionally, a keyboard, mouse or the like may be used to input data.

FIG. 7A shows a settings screen via which a user may enter subject identification data. FIG. 7B shows a setting screen for entry of monitoring settings for the subject. With particular reference to FIG. 7C, an additional settings screen is presented via which a user, such as a carer, may be able to select an option to be notified in the event that the bed is unoccupied or that a subject is at imminent risk of falling from a bed. For example, a bed fall risk alert may be triggered when the pressure sensors indicate that the subject is moving in such a way as to position themselves to climb out of bed. Accordingly, the output mechanism 180 may be configured to alert the carer of such a risk. FIG. 7D is a screenshot of a summary screen indicating the setting options selected by the user.

It is noted that alongside the bed fall risk monitoring, the system may be used to monitor bed occupancy. Accordingly, the weight exerted upon the pressure sensing apparatus may be monitored by calculating the product of the total pressure applied to the sensor and the area over which the pressure is exerted. Significant drops in the weight may be used to indicate that the bed is not occupied. A bed exit event may be recorded when the recorded weight is below a predetermined threshold, or alternatively if the recorded weight is reduced to a predetermined fraction of a previous value.

It will be appreciated that, where posture recognition is available, it may be possible to record the lack of any recognizable subject posture. Indeed, weight and posture data may be used to provide a signature of a subject such that it may be possible to identify if the occupant of the bed has changed.

The output mechanism 180 provides a channel via which the system may communicate with a user or other monitoring systems. In particular, the output mechanism may include an alarm 182, a visual display unit 184 and a transmitter 186 for relaying an output signal to a remote unit 190 for example. Other output mechanisms may include various other alarms, visual displays, audio displays, buzzers, remote units and the like, as suit requirements.

The alarm 182, such as a bell, buzzer indicator light or the like, may be used to provide a warning to alert a carer of an action required, such as the need to reposition a monitored subject, to take the subject for a walk, to prevent the subject falling from the bed or the like.

The visual display unit 184 may be used as an electronic clipboard to display various data to a user such as subject information, medical details pertaining to the patient and the like. Furthermore the visual display unit 184 may display a timer indicating time remaining until certain actions need to be performed, such as time remaining until a patient needs to be repositioned, time remaining until medicine needs to be administered, blood pressure needs to be measured, pulse monitored or the like.

In selected systems, the visual display unit 184 is operable to display a pressure map indicating current or accumulated distribution of pressure from a subject. By way of illustration only, screenshots of possible output screens are presented in FIGS. 8A-C displaying maps representing pressure data collected by the pressure sensor assembly. In particular, FIG. 8A shows a current pressure map representing the distribution of pressure from a subject being monitored in a first posture, FIG. 8B shows an alert screen indicating the need for a subject to be repositioned and FIG. 8C shows the pressure map of the subject having been repositioned into a second posture.

It is noted that in certain systems, an alert screen such as shown in FIG. 8B, may indicate regions calculated to be of particularly high risk by some indication means. So, for example, flashing colors, changed background colors or some other visual indication may be used to indicate an alert upon the pressure map. It will be appreciated that such indication may assist a carer in providing targeted care to the regions of highest need.

The processor 160 of the system control unit is configured to receive settings via the user input apparatus 120 and monitoring data via the data input 140. The processor 160 may be further configured to measure elapsed time of monitoring, typically using an internal clock. The processor 160 is operable to calculate a risk index, based upon settings and monitored data, which corresponds to the chances that a monitored subject will develop injuries such as pressure wounds. By comparing the calculated risk index with certain threshold values, the processor 160 may be operable to alert a user to necessary actions required to adequately care for a subject.

It will be appreciated that there are a plurality of factors influencing the probability that pressure wounds may develop in a subject. Monitored pressure alone may be a limited indicator of risk of developing pressure wounds because pressure wounds develop as a result of pressure being sustained over a prolonged duration. Accordingly, in certain systems, the elapsed time Δt, alone, may be used as one risk factor R₁. In other systems, an additional risk factor R₂ may be calculated from the product of pressure exerted P with the time Δt during which the pressure was recorded, such that the risk factor is given by the formula R₂=PΔt.

Optionally, in selected embodiments, the visual display unit 184 may be configured to display a map of risk indices instead of or as well as a pressure map. The risk index map may be color coded to allow a user to readily identify the areas which are at the highest risk of injury.

The displayed map may represent a two dimensional representation of a pressure sensing map with each pixel being colored according to a corresponding pressure sensing element. A carer may be able to identify thereby which areas of the subject's body correspond to the areas on the map display.

Alternatively or additionally, where possible the display may be configured to map each pixel to a point upon the body of the subject thereby indicating the risk index of each region directly on the body of a subject. It will be appreciated that such a mapping may allow the continued monitoring of the same areas of a subject even when the subject has been repositioned.

Furthermore, the threshold risk index may not be uniform for the whole body of a subject. Therefore, certain systems may be configurable such that different sets of pressure sensors have different risk index thresholds. Where appropriate, threshold values for each set of pressure sensors of a pressure sensing apparatus may be set independently by a user. It will be appreciated, however, that where possible, threshold values may be set according to body areas.

Thus in particular systems, the processors may be further configured to perform pattern recognition of the monitored subject so as to identify the various body regions and to calculate risk indices and set threshold values accordingly. This is particularly useful, for example where a sensor mat may become shifted beneath the body of a subject, without the subject's body being significantly repositioned.

More generally, however, various risk factors may be considered in the generation of a risk index, such as pressure exerted, time elapsed and sensitivity of the body tissue upon which the pressure is exerted.

The pressure risk index p(P(T,x,y)) may be considered as a function of pressure P measured at a given point (x, y) and at a given time T.

The sensitivity of the body tissue sᵤ(x, y, w, a) at a given point (x, y) may depend upon a range of medical influences, as discussed below, in particular the sensitivity may depend upon the subjects weight w and age a.

It is further noted that the pressure effect is typically time additive and this may be reflected in a calculation of overall risk index function r(τ, x, y) for example as:

$$r ( τ , x , y ) = {\sum }_{t = 1}^{τ} K ( t - τ ) * p ( P ( τ , x , y ) ) * {s}_{u} ( x , y , w , a )$$

where K(t−τ) is a time kernel function representing the additive effects of pressure.

The above described functions may be determined by experimental data. Such experimental data may be harvested, at least in part, from existing published literature. The frequency of the development of stress ulcers and the like at various areas of the body may be counted for subjects of various ages, weights, and genders as well as for different medical conditions, all of which may be included in more detailed risk factor functions. Thus a probabilistic model may be generated. Furthermore, additional data may be gathered from ongoing monitoring of subjects using pressure sensing systems. It will be appreciated that, over time, as the data reservoir grows, the accuracy of the risk factor functions may be improved.

The monitored pressure is generally measured by a pressure sensor array 220 of a pressure sensor assembly 200. Accordingly, each pixel of the pressure sensor array may correspond to a point (x, y) upon a two dimensional surface over which pressure P is measured. The sensitivity of the body tissue sᵤ(x, y, w, a) depends upon the point upon the surface of the body upon which pressure is exerted.

Where the subject is largely stationary, it may be possible to use the coordinates of the mattress to calculate the risk index for each point thereof. Accordingly, the risk index may need to be recalculated when the subject is repositioned such that a different body part comes into contact with the monitored pixel. However, where possible, it may be useful to map the coordinate system of the two dimensional surface over which pressure P is measured to the surface of the body.

In order to map the pixel measurements to the body coordinate system various techniques may be used to identify body posture of the patient or to otherwise recognize body features. Many algorithms are known in the art which may be used towards this end, such as particle component analysis, support vector machine, K-mean, two-dimensional fast Fourier analysis, earth movers distance and the like. In particular the earth mover distance (EMD) algorithm is a method to compare between two distributions, and which is commonly used in pattern recognition of visual signatures. The EMD algorithm may be readily applied, for example, to compare between a recorded posture and candidate posture types stored in a database.

The output mechanism 180 may be configured to respond to changes in monitored risk index and to alert a user appropriately. For example if a risk index exceeds a threshold value, the alarm may be sounded alerting a carer to the immediate necessity to reposition the subject. Similarly, when the risk index approaches such a threshold an alert may be displayed alerting a carer to the imminent need to reposition the subject or the like. Alternatively, different risk factors may trigger different alerts when they reach their threshold values.

With reference now to FIG. 9, a screenshot is presented of a possible output screen of a remote unit 190. The remote unit 190, such as a computer at a nurses' station in a hospital environment, may be connected conductively or wirelessly to a plurality of satellite system control units each monitoring a different subject. The display presents monitoring information relating to each of the plurality of subjects. It is noted that color or other coding may be used to show where actions are required, such as the repositioning of a subject. It is a particular feature of the remote unit that pressure maps or other data presentation relating to a plurality of subjects may be selectively presented by a common monitor.

It is noted that, where required, certain functionality, such as the option to reset the system control unit after repositioning may be disabled at the remote unit 190. This may be desirable so as to prevent the system being reset remotely without the subject actually being repositioned.

Referring now to the flowchart of FIG. 10, a possible method 600 is illustrated for use in controlling the output of such a management device. A maximum risk factor R_{MAX} is set 602, and a maximum elapsed time Δt_{MAX} is set 604, perhaps via the user input for example. The clock is started 606 and pressure data is collected by the pressure sensing apparatus 608. The processor starts calculating risk factor values 610, the calculated risk factors PΔt is compared with threshold risk factor value R_{MAX} 612 and the elapsed time Δt is compared with the maximum elapsed time Δt_{MAX} 614. If the calculated risk factor exceeds the threshold value a first alert is raised 616, such as a warning light, sound or screen notification, and if the elapsed time exceeds the maximum elapsed time Δt_{MAX} then a second alert is raised 618 such as another warning light, sound or screen notification. If the subject is repositioned, the clock may be reset and the monitoring may recommence 620.

Referring now to the flowchart of FIG. 11, the main steps of a method are presented for determining and displaying pressure related measurements for use in an injury prevention system. The method includes uses recorded pressure values from a plurality of pressure sensing elements to generate useful values of risk index and to indicate these on a map.

A risk index function is defined—step (i). The risk index function may be a simple function such as the simple accumulated pressure risk factor R₂=PΔt as described hereinabove. Alternatively, the risk index function may consider other relevant factors such as tissue type, condition of patient, region of the body and the like. Accordingly, relevant medical data pertaining to the subject may be provided to the system—step (ii).

The pressure is measured by a plurality of pressure sensing elements—step (iii). Such data may be recording using a pressure sensing mat such as described hereinabove, for example. Other pressure sensing means may be alternatively used. The time elapsed during which pressure is measured for each pressure sensing element is recorded—step (iv).

Optionally, the pressure sensing elements may be mapped directly to corresponding pixels on a two dimensional display. Alternatively, the pixel coordinates may be mapped to a body-based coordinate system—step (v). The body based coordinate system may allow the risk index to be calculated for each region of the body, which may be relevance to some defined risk index functions as described above.

A value for the risk index function may be calculated for each pixel—step (vi). It is noted that values may be calculated for each pressure sensing element in a two dimensional matrix and/or for points on the body coordinate system. The risk indices may be presented as a map—step (vii). The map displayed may provide an ongoing record of ongoing risk of a subject developing pressure related injuries which is readily accessible to a carer.

The scope of the disclosed subject matter is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

1-60. (canceled)

61. A pressure wound prevention system comprising:
at least one sensor module comprising a plurality of pressure sensor elements; and
at least one management module connectable with the sensor module, the at least one management module including:
at least one user input apparatus;
at least one output mechanism;
at least one data input connectable with the at least one sensor module; and
a processor configured to measure elapsed time, to receive pressure data from the data input, use the pressure data to calculate at least one risk index, compare the calculated risk index with at least one threshold value and to present an output via the output mechanism when the calculated risk index is greater than the at least one threshold value.

62. The pressure wound prevention system of claim 61 wherein the at least one sensor module comprises a docking station configured to connect to the at least one management module.

63. The pressure wound prevention system of claim 62 wherein the docking station includes a wireless communicator configured to communicate pressure data to the at least one management module.

64. The pressure wound prevention system of claim 61 wherein the at least one sensor module and the at least one management module each include at least one set of electrical contacts configured to connect with a corresponding set of electrical contacts on the other of the at least one sensor module and the at least one management module.

65. The pressure wound prevention system of claim 61 wherein the at least one sensor module and the at least one management module each include at least one mechanical connector configured to mechanically couple the at least one management module and the at least one sensor module.

66. The pressure wound prevention system of claim 61, wherein the at least one sensor module includes at least one layer of insulating material sandwiched between a first conductive layer and a second conductive layer.

67. The pressure wound prevention system of claim 61, wherein the at least one sensor module includes a layer of an insulating material sandwiched between a first set of parallel conductive strips and a second set of parallel conductive strips arranged orthogonally to the first set of parallel conductive strips, and a protective cover.

68. The pressure wound prevention system of claim 61, wherein the at least one sensor module includes a data storage unit configured to store historical data relating to the at least one sensor module.

69. The pressure wound prevention system of claim 61 wherein the at least one management module includes a hardware controller configured to provide analog control to the at least one sensor module.

70. The pressure wound prevention system of claim 61 wherein the at least one management module includes a system control unit configured to receive pressure data from the at least one sensor module and to present an output to a user.

71. The pressure wound prevention system of claim 61 wherein calculation of the risk index is based on:
an exerted pressure;
an elapsed time; and
medical data pertaining to a subject.

72. The pressure wound prevention system of claim 71 wherein the risk index comprises an accumulated pressure value or a product of the exerted pressure and the elapsed time.

73. The pressure wound prevention system of claim 71 wherein the output mechanism is configured to display a first warning when the calculated risk index is greater than the first threshold value, and to display a second warning when the calculated risk index is greater than a second threshold value.

74. The pressure wound prevention system of claim 61 wherein at least one set of pressure sensor elements is selected to correspond to a section of a recorded subject, and wherein the processor is configured to receive pressure data associated with each pressure sensor element, use the pressure data to calculate a plurality of risk indices, each risk index being associated with a set of pressure sensor elements, and to compare the plurality of calculated risk indices with threshold values for each set of pressure sensor elements.

75. The pressure wound prevention system of claim 71 wherein the output mechanism is operable to display a pressure map relating to one or more of the parameters selected from:
pressure readings received from the plurality of pressure sensor elements;
accumulated pressure readings received from the plurality of pressure sensor elements; and
risk indices calculated for a plurality of regions of a subject.

76. The pressure wound prevention system of claim 61 further comprising a remote unit configured to present the output to a remote user, and at least one first transmitter configured to communicate output signals to a first receiver associated with the remote unit.

77. The pressure wound prevention system of claim 76 wherein the remote unit includes the user input apparatus and a second transmitter configured to communicate output signals, and wherein the pressure wound prevention system includes at least one second receiver configured to receive input signals from the second transmitter.

78. A method for preventing the development of pressure wounds of a subject comprising the steps of:
providing a sensor module;
providing a management module comprising a processor configured to measure elapsed time and to receive pressure data from the sensor module;
calculating a risk index;
comparing the risk index with at least one threshold value; and
the processor sending an alert signal to an output mechanism if the risk index is greater than the at least one threshold value, wherein calculation of the risk index is based on:
an exerted pressure;
an elapsed time; and
medical data pertaining to the subject.

79. The method of claim 78 further comprising displaying a map of the risk index, wherein the step of calculating a risk index comprises:
defining a risk index function r(τ, x, y) relating pressure exerted upon the subject to a risk of the subject developing a pressure wound;
measuring pressure exerted by the subject upon a plurality of pixels upon the sensor module;
measuring the time elapsed during which each pixel records pressure; and
calculating the risk index function for each pixel.

80. The method of claim 79 further comprising mapping each of the plurality of pixels to a point on the body of the subject.
